(19) United States
(12) Patent Application Publication
(10) Pub. No.: US 2010/0291658 A1
(43) Pub. Date: Nov. 18, 2010

(54) PROCESS FOR SEPARATING NONPROTEINANEOUS BIOMOLECULES, IN PARTICULAR NUCLEIC ACIDS, FROM PROTEINANEOUS SAMPLES

(75) Inventors: Vera Holländer, Hilden (DE); Stefanie Schröer, Hilden (DE)

Correspondence Address: Fanelli Strain & Haag PLLC, 1455 Pennsylvania Ave., N.W., suite 400, Washington, DC, 20004, US

(73) Assignee: QIAGEN GMBH, HILDEN (DE)

(21) Appl. No.: 12/670,405

(22) PCT Filed: Jul. 25, 2008
(86) PCT No.: PCT/EP08/59774

§ 371 Date: Jun. 21, 2010

(30) Foreign Application Priority Data
Jul. 27, 2007 (DE) 10-2007035250.8

(51) Int. Cl. C07H 1/06

(52) U.S. Cl. 435/262

(57) ABSTRACT

The present invention relates to method of isolating non-protein-containing biomolecules, in particular nucleic acids, characterized in that protein degradation is carried out on a solid phase.

### FIELD OF THE INVENTION

The present invention relates to a method of separating non-protein-containing biomolecules, in particular nucleic acids from protein-containing samples, in particular from biological samples such as blood, stool, saliva, sputum or plasma.

### TECHNICAL BACKGROUND

A great many methods of separating non-protein-containing biomolecules, in particular nucleic acids from biological samples are known in the prior art.

These methods are used among other things for the purification of nucleic acids, with separation of other sample constituents such as in particular proteins and other substances that may possibly be inhibitory in subsequent applications. At the same time these methods must ensure that the nucleic acid to be isolated is not degraded enzymatically or chemically during the purification.

During the isolation of DNA, the proteins contained in the respective samples are usually broken down by lysis with a proteinase or a mixture of proteinases. During protein degradation, the DNA is usually protected against enzymatic degradation, in that the cofactors necessary for the activity of DNases are bound by chelating agents in the lysis buffer.

The isolation of RNA represents a particular challenge, as RNases are ubiquitous, are present in large amounts, are active over a wide temperature range and do not need any cofactors. Specific inactivation of all RNases during lysis is therefore impossible. Even so, for rapidly inactivating endogenous and exogenous RNases, usually lysis of the sample is carried out with very strongly denaturing lysis reagents, e.g. phenol and/or chaotropic substances.

After lysis of the sample, purification of the non-protein-containing biomolecules, in particular nucleic acids, is carried out. Purification can for example be carried out to achieve high purity, through the binding of the non-protein-containing biomolecules, in particular nucleic acids, to a solid phase, e.g. silica membranes. Such methods for e.g. nucleic acid purification are known by a person skilled in the art.

The various biological sample materials that might be of interest for isolating non-protein-containing biomolecules, in particular isolation of a nucleic acid, vary considerably with respect to their structure and composition. In particular, sample materials with an unfavorable nucleic acid/protein ratio (very much protein or very little non-protein-containing biomolecules, in particular nucleic acids) and samples containing substances that might cause interference in subsequent applications (inhibitors), impose particular demands on the method used for nucleic acid purification. These “difficult” sample materials are e.g. blood, plasma, sputum, saliva or stool.

Another particular difficulty arises when both DNA, and in a separate fraction also RNA, are to be isolated from a sample. For this purpose, protocols suitable for “lighter” sample materials are known by a person skilled in the art. These begin with strongly denaturing lysis with chaotropic reagents, to protect the RNA against degradation. Following lysis, first the DNA is bound to a solid phase. The flow-through, which contains the RNA among other things, is adjusted by adding further reagents, so that subsequently the RNA can be bound to a solid phase.

Difficult sample materials, e.g. saliva, sputum, blood or stool, which definitely require treatment with proteinases, cannot at present be used in such a method. The proteinase step necessary for complete lysis cannot be carried out at the chaotropic concentration required for protection of the RNA. Dilution to proteinase-compatible chaotrop concentrations leads on the one hand to an increased risk of RNA degradation, because RNases are also active under these conditions. On the other hand, selective binding of the DNA to a solid phase and hence separation of the various nucleic acid species are not possible from a diluted lysate. However, without proteinase treatment, the yield and quality of the isolated nucleic acid fractions are generally inadequate.

### THE PROBLEM TO BE SOLVED BY THE PRESENT INVENTION

The present invention is based on the problem of overcoming the drawbacks described above that arise from the prior art, and in particular of creating a method, for a wide range of applications, which makes it possible to isolate non-protein-containing biomolecules, in particular nucleic acids from protein-containing samples.

This problem is solved by a method as claimed in claim 1 of the present invention. Accordingly a method is proposed for isolating non-protein-containing biomolecules, in particular nucleic acids from protein-containing biological samples, comprising the steps:
a) immobilization of at least a portion of the non-protein-containing biomolecules contained in the biological sample, in particular nucleic acid(s) on a solid phase
b) enzymatic protein degradation, in which at least during a part of the protein degradation the non-protein-containing biomolecules, in particular nucleic acid(s) are bound to the solid phase.

The term “biological samples” means—but is not restricted to—in the sense of the present invention, in particular body fluids such as blood, semen, cerebrospinal fluid, saliva, sputum or urine, fluids that are obtained in the processing of blood, such as serum or plasma, leukocyte fractions or “buffy coat”, leech saliva, fecal matter, smears, aspirates, dandruff, hair, skin fragments, forensic specimens, food or environmental samples, which contain free or bound biomolecules, in particular free or bound non-protein-containing biomolecules, in particular nucleic acids, whole organisms, preferably whole non-living organisms, tissue of metazoa, preferably of insects and mammals, in particular of humans, for example in the form of tissue sections (e.g. FFPE samples), tissue fragments, or organs, isolated cells, for example in the form of adherent or suspended cell cultures, organelles, for example chloroplasts or mitochondria, vesicles, cell nuclei or chromosomes, plants, plant parts, plant tissues or plant cells, bacteria, viruses, viroids, prions, yeasts and fungi or parts of fungi. The biological samples can be fresh, or frozen, or stabilized by various methods; optionally, a suitable lysis is then carried out before step a).

The term “protein-containing” means—but is not restricted to—in the sense of the present invention, in particular that the sample contains peptides and peptide fragments, as well as whole proteins or protein complexes, which optionally can also be substituted, in particular glycosylated, phosphorylated or acetylated.

The term “non-protein-containing biomolecule” means—but is not restricted to—in the sense of the present invention, all biomolecules (except proteins), for example lipids, carbohydrates, metabolites, products of metabolism and in particular all kinds of nucleic acids.

The term “biomolecules” means—but is not restricted to—in the sense of the present invention, all molecules naturally occurring or artificially introduced in biological samples.

The term “nucleic acid” means in particular in the sense of the present invention—but is not restricted to—natural, preferably isolated linear, branched or circular nucleic acids such as RNA, in particular mRNA, siRNA, miRNA, snRNA, tRNA, hnRNA or ribozymes, DNA and the like, synthetic or modified nucleic acids, for example oligonucleotides, in particular primers, probes or standards used for PCR, nucleic acids labeled with digoxigenin, biotin or fluorescent dyes, or so-called PNAs (“peptide nucleic acids”).

The term “immobilization” means in particular in the sense of the present invention—but is not restricted to—a reversible immobilization on a suitable solid phase.

It was found, surprisingly, that even with samples in which the proportion of proteins is very much higher than the proportion of non-protein-containing biomolecules to be isolated, in particular nucleic acid(s), protein degradation can take place while the non-protein-containing biomolecules, in particular nucleic acid(s) are bound at least partially to a solid phase.

Such a device offers, for a wide range of applications within the present inventions, at least one of the following advantages:
Because during protein degradation the non-protein-containing biomolecules are bound at least partially to a solid phase, degradation of the non-protein-containing biomolecules is largely excluded or at least can largely be suppressed in most applications.
The device makes it possible to investigate samples that previously were only accessible with difficulty, such as blood, plasma, sputum, stool or saliva.
No, or only very slight, dilution occurs during protein degradation, as would be the case in protocols in solution.
By shifting protein degradation from the lysis step before the binding of the non-protein-containing biomolecules, in particular nucleic acids, to a point of time after the binding of the non-protein-containing biomolecules, in particular nucleic acids, on the solid phase, often it is also possible to carry out protocols for parallel isolation of various species, in particular nucleic acid species, from one sample.

In step a), the non-protein-containing biomolecules, in particular nucleic acids, contained in the biological sample, which essentially are completely immobilized on the solid phase, are preferred. It was found, however, that in many applications of the present invention, the method according to the invention can also be used when only a portion of the non-protein-containing biomolecules, in particular nucleic acids, is immobilized.

Accordingly, it is preferable, during step b), for the non-protein-containing biomolecules, in particular nucleic acid(s), that are bound to the solid phase, to be completely immobilized on the solid phase; however, the present invention is not restricted to this. It was found that in many applications of the present invention, the method according to the invention can also be used when, during step b), a portion of the non-protein-containing biomolecules, in particular nucleic acid(s), is detached from the solid phase or is not immobilized.

According to a preferred embodiment of the invention, the solid phase is a phase with high affinity for nucleic acids, preferably selected from the group comprising silica membranes, silica beads, magnetic particles, hydrophilic membranes, hydrophobic membranes, ion-exchange matrices, or mixtures thereof. This includes hybrid-mediated binding of nucleic acids to solid phases, for example the binding of specific nucleic acid sequences via immobilized oligonucleotides.

It was found, surprisingly, that the method according to the invention—when it is to be used for isolating nucleic acids—can be carried out not only with relatively nucleic acid-rich samples, but also with samples for which the ratio of protein to nucleic acid is unfavorable.

According to one embodiment of the invention, the ratio of protein to non-protein-containing biomolecules, in particular nucleic acids, in g/g before carrying out the method, is ≧10:1, according to another embodiment ≧100:1, according to another embodiment ≧1000:1, and according to another embodiment ≧10000:1.

According to another embodiment of the invention, step a) is carried out with addition of a chaotropic buffer. This has the advantage that possible degradation by RNases or DNases can largely be prevented beforehand, and the binding of nucleic acids in particular to silica surfaces is provided. However, other suitable lysis reagents for the particular sample material and the selected solid phase are also possible, for example the alkaline lysis of bacteria with subsequent binding e.g. to anion exchanger surfaces, known by a person skilled in the art. The buffer to be used in step a) is preferably determined on the basis of the particular sample material, the biomolecule to be immobilized, and the selected solid phase.

According to a preferred embodiment of the invention, the method additionally comprises at least one step a1), which is carried out before step b):
a1) washing of the solid phase with a solution containing at least one chaotropic substance.

This has proved favorable for many applications, because in this way gross impurities, which remain in the lysate owing to incomplete lysis without protein degradation and get onto the solid phase, can partly be removed. This removal achieved by washing is incomplete. Proteins remain on the solid phase through binding of the proteins to the solid phase itself or through binding to non-protein-containing biomolecules, in particular nucleic acids. However, washing decreases the amount of proteins to be degraded and facilitates access of the proteinase to the remaining proteins.

According to a preferred embodiment of the invention, the method additionally comprises at least one step c), which is carried out after step b):
c) washing of the solid phase with a solution containing at least one chaotropic substance.

This has proved favorable for many applications, because in this way the proteinases present in step b) can largely be inactivated. Carry-over of the enzyme into the eluate, which could prevent or disturb further enzyme-based or protein-based applications there (e.g. PCR), can thus also be prevented or largely avoided in many applications.

It will be obvious to a person skilled in the art that the method according to the invention can be followed by further steps. If DNA is to be isolated, it would for example be followed by washing with an ethanol-containing buffer, optionally drying of the membrane and elution of the DNA. In the case of isolation of RNA, it would be followed by appropriately modified steps.

Step b) can be carried out with various protein-degrading enzymes (proteases) or a mixture of several proteases. Proteinase K and QIAGEN-Protease are particularly preferred.

Step b) can optionally take place at a desired temperature during incubation. The temperature is preferably adjusted to the respective optimal temperature of the selected enzyme or enzyme mixture.

Incubation preferably takes place at a temperature from ≧0° C. to ≦100° C., preferably between ≧4° C. and ≦80° C., more preferably between ≧18° C. and ≦70° C., and especially preferably between ≧30° C. and ≦65° C.

For the case when proteinase K and/or QIAGEN-Protease are used as protein-degrading enzyme, a temperature range from ≧40 to ≦60° C., in particular ≧54° C. to ≦58° C. is preferred.

During or in step b), the protease is applied, preferably dissolved in liquid, on the solid phase.

As liquid, preferably solutions are used that produce optimal conditions for the enzymatic degradation (e.g. with respect to pH, salt compositions and concentrations) of co-factors or other conditions.

However, it was found, surprisingly, that in contrast to conventional methods of enzymatic protein degradation in solution known by a person skilled in the art, the protein degradation according to the invention can, in many applications of the present invention, take place on solid phases without special conditions provided by means of solutions. Therefore a preferred embodiment of the present invention is that step b) is carried out in water and/or in unbuffered solutions.

Solution of the protease in water is, surprisingly, sufficient in many applications to make protein degradation possible. In many applications, the solvent water and the relatively small volume thus make possible, in the method according to the invention, access of the proteases to the immobilized protein-containing sample and expression of enzymatic activity.

According to a preferred embodiment of the invention, step b) is carried out with at least one protease that has an activity of ≧1 mAU/mg.

The term “mAU” means the activity at which the enzyme releases Folin-positive amino acids and peptides equivalent to 1 μmol tyrosine per minute.

This has proved suitable for many fields of application of the present invention, because in this way good execution of the method according to the invention can often be achieved quite easily.

Preferably step b) is carried out with a (protease) activity of at least ≧10 mAU/mg, more preferably ≧20 mAU/mg and especially preferably with a (protease) activity of at least ≧30 mAU/mg.

According to a preferred embodiment of the invention, step b) is carried out for a period from

$$\geq \frac{300}{X}$$

seconds to

$$\leq \frac{2600000}{X}$$

seconds, where “X” denotes the numerical value of the (protease) activity of the enzyme used (as described above).

For the case when several enzymes are used, X means the average (protease) activity of these enzymes.

This has proved suitable in many fields of application of the present invention, because in this way on the one hand it is possible to ensure protein digestion that is as complete as possible, but on the other hand the biomolecules to be isolated are not, or are only slightly, degraded or damaged.

Preferably step b) is carried out for a period from

$$\geq \frac{450}{X}$$

seconds to

$$\leq \frac{1000000}{X}$$

seconds, more preferably

$$\geq \frac{900}{X}$$

seconds to

$$\leq \frac{108000}{X}$$

seconds, and even more preferably

$$\geq \frac{1800}{X}$$

seconds to

$$\leq \frac{54000}{X}$$

seconds.

According to a preferred embodiment of the invention, step b) is carried out for a period from

$$\geq \frac{1500}{Y}$$

seconds to

$$\leq \frac{13000000}{Y}$$

seconds, where “Y” represents the numerical value of the (protease) activity in mAU of the solution in which step b) is carried out, and where “mAU” denotes the activity at which Folin-positive amino acids and peptides are released equivalent to 1 μmol tyrosine per minute.

This has proved suitable in many fields of application of the present invention, because also in this way on the one hand it is possible to ensure protein digestion that is as complete as possible, but on the other hand the biomolecules to be isolated are not, or are only slightly, degraded or damaged.

Preferably step b) is carried out for a period from

$$\geq \frac{4500}{Y}$$

seconds to

$$\leq \frac{540000}{Y}$$

seconds, more preferably

$$\geq \frac{9000}{Y}$$

seconds to

$$\leq \frac{270000}{Y}$$

seconds.

According to a preferred embodiment of the invention, the minimum total volume during execution of step b) is adjusted so that the solid phase including all biomolecules immobilized thereon is completely wetted.

It was found that in many applications of the present invention, such wetting leads to a film of liquid that makes molecular motion and diffusion possible, to ensure access of the proteases to the proteins and the enzymatic activity.

The maximum total volume during execution of step b) is preferably adjusted so that the biomolecules to be isolated are not eluted from the solid phase or dissolved and washed away, e.g. by dripping through a membrane.

In this way, for a wide range of applications within the present invention, on the one hand it is possible to ensure that protein digestion proceeds with high efficiency, while on the other hand preventing the biomolecules that are to be isolated (in particular nucleic acids) being eluted or washed away from the solid phase during digestion.

The aforementioned components to be used according to the invention and those claimed and described in the examples are not subject, with respect to their size, form, choice of materials and technical conception, to any particular exceptional conditions, so that the criteria for selection known in the area of application can be applied without restriction.

Further details, features and advantages of the object of the invention can be seen from the subclaims and from the following description of the accompanying figures and examples, in which several examples and possible applications of the present invention are presented.

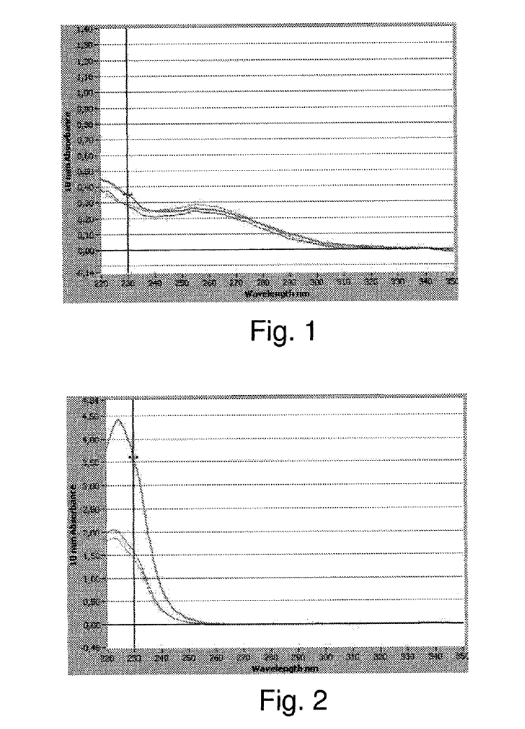

FIG. 1 shows three DNA absorption spectra after isolation using the method according to the invention in three test experiments according to example 1;

FIG. 2 shows three DNA absorption spectra after isolation in three comparative experiments according to example 1.

The invention is also explained below on the basis of examples. It is to be understood that these are provided purely for purposes of illustration and are not to be taken as any kind of restriction of the present invention, which is established exclusively by the claims.

### EXAMPLE 1

### Improvement of DNA Quality During Isolation from Saliva

For this experiment, a sample of human saliva is collected. Before collection, the test subject has not eaten or drunk anything for 1 h, to avoid contaminating the saliva sample with food residues. During sample collection, the sample is stored on ice. Next, the sample is in each case divided into 200 μl aliquots and each aliquot is mixed with 1 ml of the saliva-stabilizing solution RNAprotect Saliva from the company QIAGEN and is stored in the refrigerator for 2 days at 2-8° C. The components of the QIAamp Mini Kit and of the RNeasy Microkit from the manufacturer QIAGEN are used for the subsequent isolation of DNA and RNA from the stabilized saliva samples.

After storage, according to the manufacturer's instructions the samples are centrifuged for 10 min at 10000 rpm, the supernatant is pipetted off and the pellet is loosened somewhat by tapping on the vessel. The pellet is then dissolved in 350 μl of the GTC-containing lysis buffer RLT by vortexing. The lysate is applied on the silica membrane in the QIAamp Mini-column and is driven through the membrane by centrifugation for 1 min at 10000 rpm.

While the QIAamp Mini-column is used for DNA isolation, the flow-through is mixed with 350 μl of 70% ethanol and for RNA isolation is applied on a second silica membrane in the RNeasy Micro-column, and the RNA is isolated according to the manufacturer's instructions. The RNA was in all cases analyzed by quantitative real-time RT-PCR using the QuantiTect OneStep RT-PCR kit and primers and sample for detection of the actin β-transcript. Each sample is analyzed in duplicate. The mean value of duplicate determinations of three samples in each case is shown in Table 1.

TABLE 1

| Sample | ct-value | Mean value |
|---|---|---|
| 1a | 25.82 | 25.91 |
| | 25.59 | |
| 1b | 26.15 | |
| | 26.21 | |
| 1c | 25.75 | |
| | 25.93 | |
| 2a | 26.47 | 26.19 |
| | 26.24 | |
| 2b | 26.29 | |
| | 26.05 | |
| 2c | 26.05 | |
| | 26.01 | |

The result shows that all samples have a comparable ct-value, from which it can be concluded that all aliquots of the original saliva sample are comparable with respect to their nucleic acids.

Then the QIAamp column is used for isolating the DNA.

For samples 1a-c, for this the membrane is washed by passing through 350 μl of the guanidine hydrochloride-containing washing buffer AW1. In each case 25 μl of the proteinase K solution contained in the QIAamp Mini Kit is topped up with water to a total volume of 80 μl and applied on the membrane. After incubation for 10 minutes at 56° C., the washing step with AW1 is repeated and then it is washed with the alcohol-containing washing buffer AW2. The membrane is dried by centrifugation for 2 minutes at 14000 rpm. The DNA is eluted by applying 100 μl water and subsequent centrifugation, repeating the elution with a further 100 μl.

For samples 2a-c, the use of proteinase is omitted. The column is washed with 500 μl AW1 and directly thereafter with AW2, and then dried as for samples 1a-c and the DNA is eluted.

The quality of the DNA is determined by recording an absorption spectrum. The results are shown in FIGS. 1 and 2. FIG. 1 shows the absorption spectra of samples 1a-1c; FIG. 2 shows the absorption spectra of the comparative samples 2a-2c without use of the method according to the invention.

DNA isolation without protease (FIG. 2, samples 2a-c) shows very high absorptions in the range below 250 nm, which can be attributed to impurities. The DNA isolated using the method according to the invention (FIG. 1, samples 1a-c) is of far better quality, as the range below 240 nm only displays far less absorption and therefore is less contaminated. Moreover, a curve with a maximum at 260 nm can be seen, which can be attributed to absorption by the isolated DNA (maximum absorption of DNA occurs at 260 nm).

### EXAMPLE 2

### Improvement of DNA Yield

A saliva sample is collected, aliquoted, and stabilized as described in example 1, stored in the refrigerator for 3 days at 2-8° C. and used for RNA and DNA isolation. The DNA was eluted in 2×40 μl water. Samples 4a-c were used for DNA isolation with proteinase K digestion on the membrane according to the invention, and samples 5a-c underwent the same process without using proteinase K.

For comparison, the DNA from saliva samples is carried out by means of a method that comprises proteinase K digestion in solution. For this, as described in example 1, saliva is collected, stabilized, stored and centrifuged (samples 6a-c). After removing the supernatant, for washing the samples, 1 ml PBS is added to the pellets and mixed by vortexing. The samples are pelletized again by centrifugation for 2 minutes at 1000 rpm and the supernatant is discarded. The pellet is then dissolved in 180 μl PBS, 25 μl of proteinase K solution from the QIAamp Mini Kit (QIAGEN) and 200 μl of buffer AL are added and mixed by vortexing. The samples are incubated for 10 min at 56° C. Then each sample is mixed with in each case 200 μl of 100% ethanol and applied on the silica membrane in the QIAamp Mini-column. The further isolation of DNA is carried out as described for samples 2a-c in example 1.

The RNA was in all cases analyzed by quantitative real-time RT-PCR using the QuantiTect OneStep RT-PCR kit and primers and sample for detection of the interleukin-8 transcript. All samples showed comparable results, so that comparable NA content of the samples can be assumed (data not shown).

The DNA yield was determined by measuring the absorption at 260 nm. The mean values of the yields of samples 4, 5 and 6 (a to c) are shown in Table 2.

TABLE 2

| Sample | Yield/ng |
|---|---|
| 4a-c (according to the invention) | 602 |
| 5a-c (comparative test) | 175 |
| 6a-c (control) | 716 |

The results show that when the method according to the invention is used, far higher DNA yields can be achieved.

### EXAMPLE 3

### Downstream Analysis of the Isolated DNA

The DNA samples from example 1 were used for further analysis by quantitative real-time PCR. In addition to the samples described in examples 1 and 2, DNA isolation from the saliva sample from example 1 was carried out as a control (=sample 3a-c), as described in example 2 for samples 6a-c.

The DNA thus isolated was used in each case in duplicate determination for the detection of the gene coding for 18SrRNA. In each case 2 μl was used from samples 1 to 3, the eluates of samples 4 to 6 were diluted 1:10 with water and 2 μl thereof was used in each case. Amplification was carried out in a total volume of 25 μl with a suitable mastermix for real-time PCR, e.g. the QuantiTect SYBRGreen PCR kit from the company QIAGEN, according to the manufacturer's instructions. Amplification takes place in a suitable real-time amplifier, for example the 7700 from the company ABI. From the ct-values determined, the mean values are determined by duplicate determinations of in each case three replicates a to c and the standard deviation. The result is shown in Table 3.

TABLE 3

| | | | Standard |
|---|---|---|---|
| Sample No. | DNA sample | Mean value ct | deviation |
| 1a-c | Ex. 1 - with proteinase | 21.83 | 0.83 |
| | (according to the invention) | | |
| 2a-c | Ex. 1 - without proteinase | 27.55 | 0.15 |
| | (comparative test) | | |
| 3a-c | Ex. 1 - control only DNA | 21.41 | 0.17 |
| 4a-c | Ex. 2 - with proteinase | 17.43 | 0.34 |
| | (according to the invention) | | |
| 5a-c | Ex. 2 - without proteinase | 20.19 | 1.02 |
| | (comparative test) | | |
| 6a-c | Ex. 2 - control only DNA | 17.13 | 0.45 |

The results clearly show that without using proteinase, far poorer results are observed in PCR, but the use of proteinase K according to the invention gives good results, comparable with proteinase K in solution.

### EXAMPLE 4

### Improvement of RNA Isolation from Saliva

For this experiment, two samples of human saliva are collected, as described in example 1. Then the samples are in each case divided into 800 μl aliquots and each aliquot is mixed with 4 ml of saliva-stabilizing solution RNAprotect Saliva from the company QIAGEN and stored for a day at 2-8° C. in the refrigerator. The components of the RNeasy Microkit from the manufacturer QIAGEN are used for the subsequent isolation of RNA from the stabilized saliva samples.

After storage, the samples are centrifuged for 10 min at 10000 rpm according to the manufacturer's instructions, the supernatant is pipetted off and the pellet is loosened somewhat by tapping on the vessel. The pellet is then dissolved in 350 μl of the GTC-containing lysis buffer RLT by vortexing. The lysate is mixed with 350 μl of 70% ethanol, applied on the silica membrane in the RNeasy Micro-column and driven through the membrane by centrifugation for 1 min at 10000 rpm.

From each saliva sample, an aliquot (A) is used for RNA isolation according to the manufacturer's instructions. In each case the other aliquot (B) is used for RNA isolation by the method according to the invention. For this, the membrane is washed by passing through 350 μl of the guanidine hydrochloride-containing washing buffer RW1. In each case 20 μl of proteinase K solution is topped up to a total volume of 80 μl with water and applied on the membrane. After incubation for 10 minutes at 56° C., a washing step is carried out with a mixture of equal proportions of the lysis buffer RLT and 70% ethanol. Then washing with RW1 is repeated and then it is washed with the alcohol-containing washing buffer RPE. After another washing of the membrane with 80% ethanol, the membrane is dried by centrifugation for 5 minutes at 14000 rpm. The RNA is eluted by applying 14 μl water and then centrifuging.

The RNA thus isolated was analyzed in all cases by quantitative real-time PCR using the QuantiTect OneStep RT-PCR kit and primers and sample for detection of the IL8 transcript in triple determination. In each case 2.5 μl of the eluates was used in a total volume of 25 μl. The mean values and standard deviations obtained in the triple determinations of the samples are shown in Table 4.

TABLE 4

| | | | Standard |
|---|---|---|---|
| Sample No. | RNA sample | Mean value ct | deviation |
| 1A | without proteinase | 28.35 | 0.27 |
| | (comparative test) | | |
| 1B | with proteinase | 25.43 | 0.23 |
| | (according to the | | |
| | invention) | | |
| 2A | without proteinase | 31.45 | 0.16 |
| | (comparative test) | | |
| 2B | with proteinase | 27.14 | 0.57 |
| | (according to the | | |
| | invention) | | |

The results clearly show that also in the isolation of RNA from protein-rich samples without using proteinase, far poorer results are observed in PCR, but the use of proteinase K according to the invention gives far better results.

### EXAMPLE 5

### Improvement of Downstream Analyses of DNA from FFPE Samples

This experiment uses tissue samples (FFPE samples) from rat liver fixed in formaldehyde solution and embedded in paraffin. Sections approx. 20 μM thick are prepared from these samples using a microtome, and 1 section is used per sample. Components of the RNeasy FFPE kit, of the Allprep DNA/RNA kit and of the QIAamp Mini Kit from QIAGEN are used for the subsequent isolation of DNA from the FFPE sections.

The tissues are deparaffined—by washing with 1 ml xylene—according to the manufacturer's instructions (manual of the RNeasy FFPE kit) and, after centrifuging the sample and removing the supernatant, 1 ml of 100% ethanol is added. After centrifuging the sample again and removing the supernatant, the samples are dried for 10 min at 37° C. Then, after adding 150 μl of the buffer PKD and 10 μl of proteinase K from the RNeasy FFPE kit, the samples are incubated for 3 h min at 56° C. and 15 min at 80° C. After adding 320 μl of the chaotrop-containing lysis buffer RBC, the resultant mixture is applied on the silica membrane in the Allprep DNA column (from the Allprep DNA/RNA Mini Kit) and is driven through the membrane by centrifugation for 1 min at 14000 rpm.

Then, for one sample (sample 1) a proteinase K treatment is carried out on the silica membrane, by applying 20 μl proteinase K and 60 μl water on the membrane and incubating the sample for 10 min at 56° C., whereas the comparative sample (sample 2) is not treated with proteinase K. The membranes of both samples are then washed by passing through 500 μl of the guanidine hydrochloride-containing washing buffer AW1 and then 500 μl of the alcohol-containing washing buffer AW2. The membrane is dried by centrifugation for 2 minutes at 14000 rpm. The DNA is eluted by centrifugation by applying 30 μl water after incubation for one minute.

The DNA thus obtained is first checked by agarose-gel electrophoresis. For this, in each case 5 μl of the eluate obtained is diluted with 15 μl water and separated on a 0.5% agarose-TAE-gel for approx. 4 h at 60 V. The gel stained with ethidium bromide shows in both cases fragmented DNA, recognizable from a light “smear” distributed over a certain size range, with the comparative sample (2) essentially containing smaller fragments than sample 1 after proteinase treatment on the membrane. Use of the method according to the invention therefore leads to the isolation of larger DNA fragments.

The DNA that can be isolated from FFPE samples is always fragmented, as the DNA is already degraded during fixing, embedding and storage. In addition, as a result of fixing in formaldehyde solution, the DNA is covalently crosslinked with other nucleic acids and primarily with proteins, which makes both the isolation of the DNA, and analysis of the DNA by amplification techniques, extremely difficult. Now to investigate the effect of the method according to the invention not only on the isolation of DNA, but also on analysis by amplification, the eluates were used for further analysis by quantitative real-time PCR.

The isolated DNA was used in each case in duplicate determination for the detection of a 465 bp amplicon of the gene coding for the prion protein.

The eluates were in each case diluted 1:20 with water and 5 μl of these dilutions was used in real-time PCR. Amplification was carried out in a total volume of 25 μl with a suitable mastermix for real-time PCR, e.g. the QuantiTect SYBRGreen PCR Kit from the company QIAGEN, according to the manufacturer's instructions. Amplification was carried out in a suitable real-time amplifier, for example the 7700 from the company ABI. The mean values from the duplicate determinations of the duplicate and the standard deviation were determined from the ct-values found. The result is shown in Table 5.

TABLE 5

| | | | Standard |
|---|---|---|---|
| Sample No. | DNA sample | Mean value ct | deviation |
| 1 | with proteinase | 26.20 | 0.26 |
| | (according to the | | |
| | invention) | | |
| 2 | without proteinase | 29.06 | 0.20 |
| | (comparative sample) | | |

The results clearly show that the use of proteinase K according to the invention gives far better results than a comparable assay without the method according to the invention. This is particularly surprising, because both the comparative sample, and the sample treated by the method according to the invention, underwent a three-hour treatment with proteinase K in solution at the start of the DNA isolation process. Nevertheless, the very short treatment time of 15 min, compared with this, produced an improvement in DNA fragment size and in particular an improvement in the amplifiability of the DNA.

1. A method of isolating non-protein-containing biomolecules from protein-containing biological samples, comprising the steps:
a) immobilization of at least a portion of the non-protein-containing biomolecules, contained in the biological sample, on a solid phase
b) enzymatic protein degradation, wherein during the protein degradation, the non-protein-containing biomolecules, in particular nucleic acid(s) are bound to the solid phase.

2. The method as claimed in claim 1, wherein the ratio of protein to non-protein-containing biomolecules, in particular nucleic acids in g/g in the biological sample, before carrying out the method, is ≧10:1.

3. The method as claimed in claim 1, wherein the non-protein-containing biomolecules comprise nucleic acids.

4. The method as claimed in claim 1, wherein in that the solid phase is a phase with high affinity for nucleic acids, preferably selected from the group comprising silica membranes, silica beads, magnetic particles, hydrophilic membranes, hydrophobic membranes, ion-exchange matrices, or mixtures thereof.

5. The method as claimed in claim 1, additionally comprising a step a1), which is carried out between step a) and b):
a1) washing of the solid phase with a solution containing at least one chaotropic substance.

6. The method as claimed in claim 1, additionally comprising a step c), which is carried out after step b):
c) washing of the solid phase with a solution containing at least one chaotropic substance.

7. The method as claimed in claim 1, wherein step b) is carried out with at least one protease, which has an activity of ≧1 mAU/mg.

8. The method as claimed claim 1, wherein in that step b) is carried out for a period from
$$\geq \frac{300}{X}$$
seconds to
$$\leq \frac{2600000}{X}$$
seconds, where “X” denotes the numerical value of the protease activity of the enzyme used.

9. The method as claimed in claim 1, wherein step b) is carried out for a period from
$$\geq \frac{1500}{Y}$$
seconds to
$$\leq \frac{13000000}{Y}$$
seconds, where “Y” denotes the numerical value of the protease activity in mAU of the solution in which step b) is carried out.

10. The method as claimed in claim 1, wherein step b) is carried out in water and/or in unbuffered solutions.
